(19) United States
(12) Patent Application Publication
(10) Pub. No.: US 2003/0064059 A1
(43) Pub. Date: Apr. 3, 2003

(54) Embryonic stem cells, clinical applications and methods for expanding in vitro

(75) Inventors: William C. Rader, Malibu, CA (US); Albert Scheller, Tegernsee (DE)

Correspondence Address: CHRISTIE, PARKER & HALE, LLP, P.O. BOX 7068, PASADENA, CA, 91109-7068, US

(73) Assignee: Medra, Inc.

(21) Appl. No.: 10/280,791

(22) Filed: Oct. 25, 2002

Related U.S. Application Data
(62) Division of application No. 09/907,790, filed on Jul. 18, 2001.

(51) Int. Cl. A61K 48/00; C12N 5/08

(52) U.S. Cl. 424/93.21; 435/368; 435/372

(57) ABSTRACT

The present invention provides a method for the in vitro culture of embryonic stem cells, wherein the stem cells continue to express no antigen or antigen CD117, and mostly remain undifferentiated during culture. The present invention also relates to purified preparations of embryonic stem cells and for uses of embryonic stem cells in treating a wide variety of conditions, diseases and disorders.

### CROSS-REFERENCE TO RELATED APPLICATION

This is a division of Ser. No. 09/907,790, filed on Jul. 18, 2001, entitled EMBRYONIC STEM CELLS, CLINICAL APPLICATIONS AND METHODS FOR EXPANDING IN VITRO, the contents of which are hereby expressly incorporated herein by reference.

### FIELD OF INVENTION

This invention relates to the in vitro expansion of undifferentiated embryonic stem cells, to purified cultures of expanded embryonic stem cells, and to the use of embryonic stem cells in the treatment of a wide variety of diseases, conditions and disorders.

### BACKGROUND OF THE INVENTION

Shortly after fertilization, a mammalian egg begins to divide into identical, totipotent cells. Each of these cells, if isolated, has the potential to develop into a fetus. Within a very short period of time, however, these cells begin to form into a hollow ball of cells called a blastocyst. The outer layer of the blastocyst will ultimately give rise to the placenta and other tissue necessary to support fetal growth. Inside this outer cell layer is a cluster of cells, the inner cell mass, which will give rise to the cells of the fetus. These cells are pluripotent stem cells, which, although having the potential to develop into many types of cells, no longer have the potential to develop into a fetus if isolated.

The pluripotent stem cells can further specialize into stem cells committed to develop into particular cell types. For example, hematopoietic stem cells will give rise to red blood cells, white blood cells, and platelets, while neuronal stem cells will give rise to the various types of nerve cells.

Embryonic stem cells have no antigenicity and thus are well-suited for therapies involving introduction of stem cells into the human body. Although human embryonic stem cells can be isolated from aborted fetuses and/or embryos produced by in vitro fertilization techniques on an as-needed basis, alternate sources, such as cultured embryonic stem cell lines, are preferred both for ethical and economic reasons. For example, the number of embryonic stem cells used for treating patients according to the practice of the instant invention requires the use of a single fetus per patient as a source of the cells. By contrast, expanding a population of embryonic stem cells while maintaining the cells in an undifferentiated and pluripotent state would allow several thousand patients to be treated with cells isolated from a single fetus.

Recently, methods for culturing pluripotent human stem cells in vitro have been developed by James Thomson and Michael Shamblott. James Thomson, et al. (1998) Embryonic stem cell lines derived from human blastocysts, Science 282: 1145-1147; Michael J. Shamblott, et al. (1998) Derivation of pluripotent stem cells from cultured human primordial germ cells, Proceedings of the National Academy of Sciences 95: 13726-13731. Thomson isolated pluripotent cells from the inner cell mass of blastocysts, while Shamblott isolated the pluripotent stem cells from fetal tissue obtained from terminated pregnancies. In both cases, the cells must be grown on a layer of cultured mouse fibroblast feeder cells, a potential source of contamination should these cells be used to treat human (or veterinary) patients.

What is needed, therefore, is a method for expanding embryonic stem cells in vitro that does not require such feeder cells and which maintains the stem cells in their undifferentiated and pluripotent state, thereby reducing the number of embryos or fetuses required for stem cell therapy.

### SUMMARY OF THE INVENTION

The present invention is for an in vitro culture of embryonic stem cells, wherein the stem cells continue to express no antigen or antigen CD117, and mostly remain undifferentiated during culture. The present invention also relates to purified preparations of embryonic stem cells and for uses of embryonic stem cells in treating a wide variety of conditions, diseases and disorders.

### BRIEF DESCRIPTION OF THE DRAWINGS

FIGS. 1a, 1b and 1c are drawings of a device useful for collecting and removing cells from a liquid medium.

FIG. 2 is a photograph illustrating the migration of stem cells toward stressed or cancer cells.

### DETAILED DESCRIPTION OF THE INVENTION

The process of this invention provides a relatively simple and efficient method for expanding undifferentiated and pluripotent embryonic stem cells in culture while maintaining the pluripotent and undifferentiated state of the cells. According to practice of the invention, embryonic stem cells are expanded without the use of feeder cells such as mouse fibroblast cells and remain undifferentiated even after 10 to 14 cell cycles. As a result, sufficient embryonic stem cells may be produced to treat thousands of patients from a single aborted fetus, greatly reducing the costs of such procedures and minimizing the number of aborted fetuses required for treatment of patients.

This invention also provides an apparatus and a method for isolating and collecting cells from cell cultures, and a method for identifying and diagnosing tumors and sites of suspected tissue damage using embryonic stem cells.

Finally, this invention provides a method for treating a variety of diseases, including cancer, AIDS, and genetic disorders such as hemophilia and sickle cell anemia, using embryonic stem cells.

The procedure for isolating and propagating the embryonic stem cells is given in detail below.

Embryonic stem cells may be isolated from a number of sources. One source of stem cells is an aborted fetus that has been pre-screened for a variety of biological agents and/or genetic conditions. Preferably, the fetus is pre-screened for Mycoplasma incognitus, hepatitis B, hepatitis C, and HIV, using standard protocols. If possible, the mother is also tested for the same biological agents. Polymerase chain reaction (PCR) assays, using standard protocols known to those skilled in the art, are a preferred means of screening fetuses as such assays are relatively rapid and cost-effective while extremely sensitive and reliable.

Once an aborted fetus (or “abortus”) is found to be free from undesirable biological agents, the embryonic stem cells are extracted by standard protocols. In one embodiment, the embryonic stem cells are extracted from the abortus and processed through a series of filtration steps. Typically, between 10⁵ and 10⁸ human stem cells are isolated from a single abortus. Hematopoietic and neuronal stem cell are isolated separately by manual separation and collection under the microscope. Both cell types are collected at the same time.

An alternative source of embryonic stem cells are fresh or frozen cleavage stage embryos, produced by in vitro fertilization for clinical purposes and donated by informed consent. Such embryos are cultured to the blastocyst stage, wherein the inner cell masses (containing the stem cells) are isolated as described in Thomson et al. (1998) Science 282: 1145-1147, and U.S. Pat. No. 5,843,780, both hereby incorporated by reference in their entirety.

Once isolated, the embryonic stem cells are introduced into an embryonic cell culture medium comprising a supplemented Dulbecco's Modified Eagle's Medium (DMEM), such as Mesenchymal Stem Cell Basal Medium (MSCBM), fetal bovine serum or other stem cell media, L-Glutamine, antibiotics, and amino acids. In addition, the medium may be supplemented with growth factors, such as Leukemia Inhibitory Factor (LIF), Fibroblast Growth Factor (FGF), Neuronal Growth Factor (NGF), Mesenchymal Growth Factor (MGF), Platelet Derived Growth Factor (PDGF) or Insulin-like Growth Factor (IGF). In a preferred embodiment, the medium is supplemented with LIF, FGF, NGF and MGF. The pH of the embryonic cell culture medium is adjusted with NaOH to between about 6.5 and 7.8.

The embryonic stem cells are then placed in a 5% CO₂ incubator and grown at about 34° C. to about 42° C., preferably about 37° C., until the desired cell density is reached, generally about five days.

During incubation, a microcurrent is applied to the culture media. The amount of current applied ranges from about 0.1 μAmp to about 1 mAmp, preferably between about 1 μAmp and about 100 μAmp, with a frequency range from about 0.1 Hz to about 100 Hz, preferably between about 1 Hz and about 50 Hz.

Application of the microcurrent may be continuous or intermittent, with the duration varying according to the microenvironment of the cells. The duration of the microcurrent ranges from about 200 milliseconds, applied once per day, to the continuous application of microcurrent over the entire period of incubation. Preferably, the microcurrent is applied for as short a duration as necessary to maintain the cells in their pluripotent and undifferentiated state, as prolonged application of microcurrent may affect the pH of the media, adversely affecting cell viability.

During incubation, the cell cultures are tested daily for cell count, cell viability and pH, using standard laboratory procedures. It is important to ensure that the pH of the medium stays between about 6.5 and 7.8, as cell viability may decrease outside of this range. The pH of the medium may be maintained within this range by the addition of buffers, replacing the medium with fresh medium, and altering the microcurrent.

Incubation continues until the desired cell density is achieved. The duration of incubation will vary, depending on the growth rate of the cells, which, in turn, is dependent on the microenvironment in which the cells are grown. Typically, after incubation for about five days as described above, the embryonic stem cell density is between about 10⁷ and about 10⁸ cells per cubic centimeter, with a cell viability between about 90% and about 99%.

The cells are then scraped from the inner surfaces of the flasks and aseptically collected according to standard protocols. The cells are separated from the liquid medium by centrifugation under conditions sufficient to pellet the cells. The supernatant is discarded and the cell pellet is gently resuspended in a buffer containing 5% to 20% dimethyl sulfoxide (DMSO).

Alternatively, the cells may be separated from the liquid medium by filtration through a filter membrane having a porosity of about 0.2 μM. The cells retained on the filter are rinsed from the filter into a storage receptacle with a buffer containing DMSO or other similar material. In order to capture cells which may pass through the membrane as filtrate, and to thereby maximize recovery of the cells, the suspended cells may be passed through stacked filter membranes. However, this technique typically requires the application of pressure or a vacuum, which may damage the cells, reducing viability.

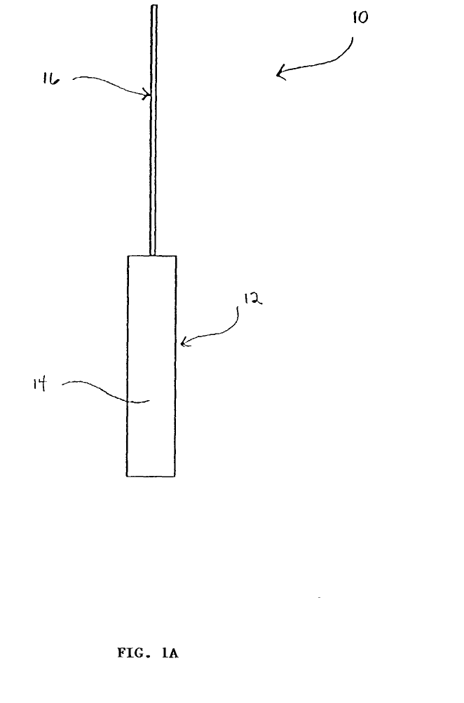

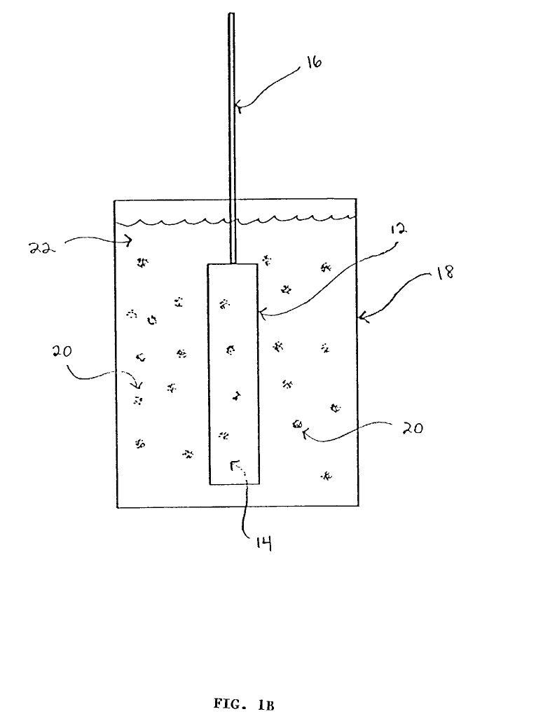

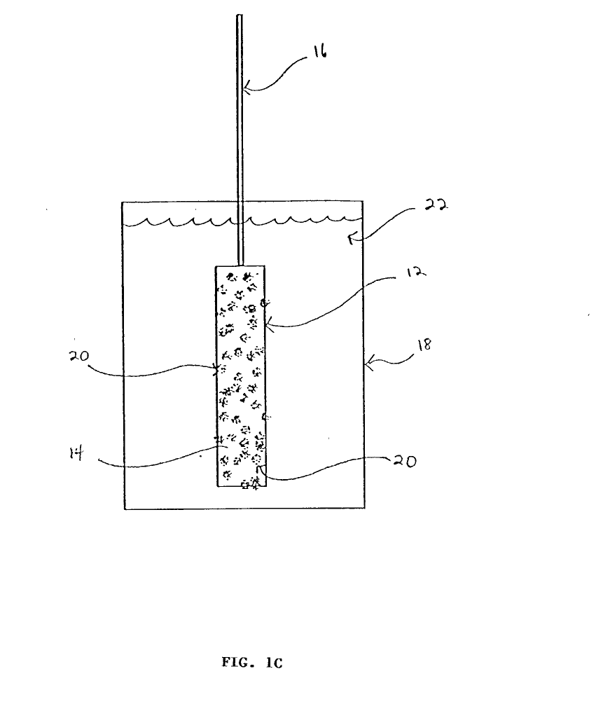

As an alternative to centrifugation or filtration as a means to collect the cells, a collection rod, shown in FIG. 1, may be used. The collection rod takes advantage of the membrane potential of the cells, which gives living cells a positive charge on the cell surface, and provides an efficient and gentle method for collecting cells with minimal cell damage.

As shown in FIG. 1A, the collection rod 10 comprises a cylinder 12 and a handle 16, attached to one end of the cylinder. The cylinder is composed of metal or other material that conducts electricity. Alternatively, the cylinder may be composed of a non-conductive material, such as porcelain, coated with a conductive material. The handle 16 is composed of a non-conductive material such as plastic or rubber.

As shown in FIG. 1B, the collection rod is inserted into a container 18 containing cells 20 in a liquid medium 22. An electrical current from about 1 μAmp to about 10 mAmp is then applied to the cylinder such that the cylindrical surface 14 is negatively charged. Living cells, which have a membrane potential between about −200 mV and 0 mV, most typically around −40 mV, are positively charged on the cell surface. The cells are thus attracted to the negatively charged cylindrical surface, where they accumulate and attach without any application of pressure. As cells accumulate on the cylindrical surface, as shown in FIG. 1C, it may be necessary to increase the negative charge on the cylindrical surface to compensate for the build-up of positively charged cells.

After sufficient cells have accumulated on the cylindrical surface, the collection rod is removed from the container while maintaining the charge to the cylindrical surface. The collection rod, with attached cells, is inserted into an appropriate receptacle and the polarity of the charge to the cylinder is reversed so that the cylindrical surface is now positively charged, causing the positively charged cells to detach from the cylindrical surface into the receptacle. A small volume of liquid, such as a buffer or DMSO-containing buffer, may be used to rinse the cells from the cylindrical surface and to collect the cells in the receptacle.

After collection, the cells are frozen and stored at from about −20° C. to −200° C. Generally, the cells are divided into aliquots of between about 0.5 mL and 2.0 mL prior to freezing.

In the above-described embodiment, a microcurrent is applied to the medium during incubation to maintain the cultured embryonic stem cells in an undifferentiated and pluripotent state. In an alternative embodiment, the embryonic stem cells are grown under hyperthermic conditions (i.e., at temperatures above normal growth temperatures) to maintain the undifferentiated and pluripotent state. In this embodiment, the cells are grown at about 42° C. in a supplemented DMEM, such as MSCBM containing fetal bovine serum or other stem cell media, L-Glutamine, antibiotics, and amino acids, until the desired cell density is reached. Under hyperthermic conditions, the embryonic stem cells maintain their pluripotent and undifferentiated state without application of a microcurrent, even in the absence of growth factors, although the application of a microcurrent as described above may improve growth. It is not known why hyperthermic growth conditions inhibit differentiation, although the induction of heat shock proteins may play a role.

After expansion, the cells may be subjected to a variety of quality control tests to ensure that the cells are viable, uncontaminated, undifferentiated and pluripotent. For example, the cells are visually inspected for morphological characteristics of differentiated cells. Also, the samples of the expanded cells may be tested for cell count and viability, and may be tested for the presence of Mycoplasma incognitus, hepatitis B, hepatitis C, and HIV, as described by Kaneko, et al. (1990) Gastroenterology 99: 799; Liang, et al. (1990) Hepatology 12: 202; Isopet, et al. (1999) J. Med. Virol. 58:139 and Preugschat, et al. (2000) Biochemistry 39: 5174, all hereby incorporated by reference in their entireties.

The cell samples may also be used in an Enzyme-Linked Immunosorbent Assay (ELISA) to test for the presence of CD117, a stem cell factor receptor specific to stem cells. Human Soluble CD117 ELISA kits are available from Appledale (Essex, U.K.) or from Diaclone (Besancon Cedex, France). The presence of the CD117 receptor provides a measure of the viability of hematopoietic, myelopoietic (mesenchymal, fibroblast) and neuronal stem cells. Other receptors may also be used. For example, the presence of NGF is indicative of neuronal stem cells.

Embryonic stem cells isolated from individual embryos and/or expanded according to the practice of the invention may be used in a variety of therapeutic and diagnostic modalities. Embryonic stem cells have the developmental potential to give rise to any differentiated cell type. Thus, a disease that results from the failure, either genetic or acquired, of specific cell types is potentially treatable by transplantation of embryonic stem cells or cells derived from embryonic stem cells. Thus, for example, embryonic stem cells may be used to treat genetic disorders such as sickle cell anemia, hemophilia and other hematologic disorders, heart disease, autoimmune diseases and metabolic disorders. Similarly, embryonic stem cells may be used to improve healing of fractures, increase cognitive abilities, and improve muscle and skin tone.

In the practice of the present invention, both hematopoietic stem cells and neuronal stem cells are administered to a patient in need thereof to treat a wide variety of disorders or diseases. Approximately 10 million to about 100 million hematopoietic stem cells are diluted in distilled water to a final volume of 6 cubic centimeters and administered by intravenous injection. Similarly, about 10 million to about 80 million neuronal stem cells are diluted in distilled water to a final volume of 6 cubic centimeters and administered subcutaneously. Typically, a patient only requires one dose of each type of stem cell. Alternatively, the embryonic stem cells may be administered intrathecally, by direct injection, as into bone marrow or to the retro bulbar portion of the eye, or intralesionally, as in a cell paste or gel.

Optionally, injection of stem cells may be accompanied by application of fetal or embryonic thymus subcutaneous injections or sheep thymus subcutaneous injections. The fetal thymus provides growth factors and hormones. The sheep thymus stimulates the immune system, as well as the fetal thymus, resulting in an expression of different interleukins of both fetal and sheep thymus.

In the case of patients suffering from a genetic disorder, embryonic stem cells are administered to the patient by both intravenous injection (hematopoietic cells) and subcutaneous injection (neuronal cells). These embryonic stem cells carry the desired genetic trait and, once administered, differentiate to provide the patient with a population of cells expressing the heretofore lacking gene product.

Another example of the therapeutic use of embryonic stem cells is the use of stem cells containing (or lacking) specific chemokine receptors for the treatment of patients with AIDS. Research into the mechanism of HIV transmission has found that the incidence of transmission of HIV between active sexual partners who are not using any form of prophylactic is greatly reduced when the non-infected sexual partner is homozygous for the genetic marker CCR5. Similar results have been found in CCR5⁻/CCR5⁻ (CCR5-def) babies born to HIV-infected mothers.

CCR5 chemokine receptors are G-proteins found in the cell membranes of activated T-cells, monocytes, macrophages and dendritic cells. The receptors not only bind chemokines, but have also been found to act as co-receptors for the binding of immunodeficiency viruses. Macrophage-tropic HIV strains, which infect macrophages more readily than they infect CD4+ T cells, use the CCR5 chemokine receptor on the surface of macrophages in conjunction with the CD4 receptor to enter and infect a cell. Experimental evidence suggests that macrophage-tropic HIV strains establish the HIV infection, while CD4+ T cell-tropic strains are crucial in later stages of the infection. Thus, macrophages defective for CCR5 do not readily bind macrophage-tropic HIV strains, and individuals carrying a homozygous CCR5-def mutation are resistant to the initial HIV infection.

Thus, individuals whose macrophages and T cells are deficient for CCR5 are particularly resistant to infection by HIV. Accordingly, embryonic stem cells found to be CCR5 homozygous deficient are isolated, then are given to a patient via intravenous injection, along with embryonic thymus given in a subcutaneous injection and sheep thymus in a subcutaneous injection. Some portion of the injected embryonic stem cells will differentiate into macrophage and T cells, deficient in CCR5, providing the recipient of the injection with a population of cells resistant to infection by immunodeficiency viruses such as HIV. Optionally, injection of these expanded embryonic stem cells may be preceded by aplasia of the recipient's bone marrow using conventional techniques. The GP41 and the NEF fragment may be important for the inhibition of the polymerase for the virus. It affords the possibility the patient cannot reduplicate the virus (stops viral replication).

Hematopoietic embryonic stem cells may also be used topically to treat skin debridements and other forms of tissue damage. In one embodiment, the hematopoietic stem cells may be mixed with a biocompatible carrier, such as a gel, ointment or paste, and applied to damaged skin or mucosal tissue to accelerate healing as well as to heal wounds resistant to healing, such as diabetic or decubitus ulcers. Alternatively, the cells may be provided in a suspension or emulsion. Preferably, the carrier also contains antimicrobial agents, analgesics, or other pharmaceutical agents. A carrier suitable for applying stem cells comprises dermatan sulfate, hyaluronic acid, chondroitin sulfate, and collagen. Applied intralesionally, the embryonic stem cells will differentiate and eventually replace the damaged cells.

Alternatively, large quantities of embryonic stem cells can be expanded according to the practice of the present invention and induced to differentiate in vitro into skin with no antigenicity, which can then be stored for long periods in liquid nitrogen until required for use as a graft for burn victims or to treat decubital ulcers, ulcers secondary to diabetes, etc. Cultured embryonic stem cells may be induced to differentiate into skin cells by the addition of cells of stratum granulosum, for example, to the culture medium. Other appropriate additives include fibroblasts, fibroblast growth hormone and calcium.

Further, embryonic stem cells expanded according to the practice of the present invention can be induced to differentiate in vitro by altering the microenvironment of the cells by, for example, the addition of appropriate growth factors. These differentiated cells can then be administered to the site of tissue damage. Thus, for example, embryonic stem cells may be induced to differentiate into cardiac muscle cells by the addition of cardiac muscle cells and mesenchymal growth factor to the culture medium, then administered to a patient with damaged cardiac muscle via intravenous injection. Some portion of the differentiated cells will be incorporated into the patient's cardiac tissue, reproducing and repairing the damaged muscle.

In another example, embryonic stem cells can be used to generate new hair growth. Using embryonic stem cells, it is possible to pre-determine the hair distribution as well as the number of hairs per given area (thickness). Also, the hair produced can be of any color or hair type, depending on the particular cells used. Following treatment, the new hair will grow naturally. Embryonic stem cells can be used to grow hair in locations other than the scalp, including grow eyelashes, eyebrows, or even pubic hair.

According to this process, at least one hair, including the hair follicle, is taken from the client, or if the client desires a different hair type (straight, curly, other color), is taken from a different source within an inventory of hairs. The hair is placed in a growth factor-supplemented culture medium along with embryonic stem cells. The microenvironment of the hair follicle triggers differentiation of the embryonic stem cells into hair cells, creating new hair follicles.

In one embodiment, computer animation is used to allow the recipient of the new hair to experiment with different hair types and distributions, to determine how they would like their hair to look; i.e., a filled-in bald spot, a whole head of hair, thickness, type of brow line, straight or curly hair, etc. Once the desired hair distribution is determined, a scalp cap is prepared containing specialized needles, each with a micropore that allows only a single cell to enter the scalp at the site of the needle. These needles are composed of an inert material such as titanium, porcelain, or other inert material. The client predetermined computer program selection controls the placement of the specialized needles in the cap.

The scalp is then sprayed with a local anesthetic. The cap is placed over the client's head and the differentiating embryonic stem cells are delivered to the scalp through the micropore needles, a single cell delivered through each needle. Within two weeks, the first hairs appear. These are called Lanugo hair and are exactly the same hair as that is found in a newborn baby. Within approximately one month, these hairs fall out (as occurs in the newborn) and the permanent hair begins to grow in. The rate of the new hair is faster than the natural growth of hair (approximately 1 cm per month). The new hair has the same qualities (strength, look, etc.) as an adolescent's hair and continues to grow normally.

In still another example, neuronal stem cells cultured in a gel differentiated into a nerve cell 2.5 inches long. Nerve tissue grown in gels may be applied directly to damaged nerves, reconnecting severed nerve cells.

In one embodiment, a tube made from omentum is filled with a gel suitable for culturing neuronal stem cells, as is known in the art, and neuronal stem cells are added. The neuronal stem cells differentiate into nerve cells within the omentum, which is then implanted into the body at the site of nerve damage; that is, between the two ends of a severed nerve.

After implantation, a di-polar current (positive on one side, negative on the other) is applied. To produce an afferent nerve, the positive current is applied near the lesion between the implant and the patient's brain. Similarly, to create an efferent nerve, a positive current is applied near the lesion between the implant and the peripheral nerves. The current used to produce a nerve depends on the distance of the lesion.

Alternatively, embryonic stem cells may be injected directly into sites of tissue or organ damage, where the microenvironment of the surrounding tissue will induce differentiation of the injected cells. For example, mesenchymal stem cells injected into cardiac muscle will differentiate into cardiac muscle cells. Thus, these cells can be used to treat heart damage following, for example, a myocardial infarction.

It has been found that embryonic stem cells, when placed in an environment where they can migrate among other cells, preferentially accumulate near stressed cells, such as cells with damaged membranes, or cancer cells. This characteristic suggests that embryonic stem cells, injected intravenously or subcutaneously, may target sites of cell damage, allowing the injected embryonic stem cells to accumulate at and repair damaged tissue.

It is not completely clear how embryonic stem cells detect and distinguish between stressed and unstressed cells, or between cancer and noncancer cells, but the reduced membrane potential of the cancer cells and cells with damaged membranes from other traumatic, hypoxic or diseased states may play a role. It is known, for example, that cancer cells have a lower membrane potential, typically from about −40 mV to about 0 mV, than non-cancer cells. Embryonic stem cells, administered to a patient having a tumor or other site of cell damage, detect the lower membrane potential of the cancer and/or damaged cells, preferentially migrating and accumulating at the site of the tumor or other cell damage.

Whatever the exact mechanism, this characteristic of embryonic stem cells also makes them useful as a diagnostic reagent. For example, embryonic stem cells may be labeled with a biocompatible agent such as technetium, indium, iodium and the like, then administered to a patient displaying symptoms of an unknown etiology or to detect an asymptomatic underlying or latent disease process. After allowing sufficient time for the administered cells to circulate through the body, the patient may be examined to detect the specific label used on the cells and to thereby determine sites of accumulated labeled cells. Such sites should be considered potential areas of tissue damage, tumor growth, or undiscovered potential disease processes.

Moreover, this characteristic also makes embryonic stem cells extremely useful for site-specific delivery of drugs. For example, embryonic stem cells known to be toxic or otherwise harmful to cancer cells may be administered, alone or in combination with aplasia of the bone marrow, to cancer patients. The embryonic stem cells will accumulate at the site of the tumor, eventually releasing, or expressing, the cytotoxic agents and destroying the cancer cells. For example, embryonic stem cells can be modified with specific genetic information, such as “suicide bags” with chemotherapeutic agents, caspase, or other apoptosis inductors. Alternatively, embryonic stem cells may be selected that express antiangiogenic factors, such as marimastate, batimastate, vascular endothelial growth factor (VEGF) or endostatin, or that contain metabolic compounds that can kill cancer cells (i.e., dystrophin) such as lactate-shuttle inhibitors. Such “suicide bags” can be lysosomes or a special coated compound.

Alternatively, embryonic stem cells may be administered to supplement conventional chemotherapy treatment of cancer patients. In conventional chemotherapy, cytostatic agents are administered to destroy cancer cells. However, cytostatic agents do not distinguish between normal cells and cancer cells, and may destroy the patient's noncancer cells, including the cells of the patient's immune system. As a consequence, while undergoing chemotherapy, and for some period after the chemotherapy stops, cancer patients are susceptible to infection due to their compromised immune system. By administering embryonic stem cells to patients undergoing chemotherapy, however, some portion of the injected cells will differentiate into a new immune system, replacing white blood cells, red blood cells, platelets and other cells destroyed by chemotherapy.

Still another use for embryonic stem cells is for developing and testing new drugs. For example, embryonic stem cells may be expanded according to practice of the present invention and used as a substrate for testing the safety of new pharmaceuticals.

In addition, embryonic stem cells can be used to produce human proteins for therapeutic use. For example, a cell line of embryonic stem cells producing the blood clotting protein Factor VIII has been identified. This cell line can be maintained in culture and expanded according to practice of the invention to provide a source from which human Factor VIII can be isolated.

The above discussion has focused on methods for expanding and using human embryonic stem cells. However, these methods and uses apply to other mammalian embryonic stem cells as well. Embryonic stem cells can be isolated from the fetuses of other mammalian species and used in a variety of veterinary applications. For example, embryonic stem cells isolated from horse fetuses could be administered to horses used in racing to improve muscle tone, strength and stamina, and to increase reflexes, or administered to family pets to treat diseases such as cancer or to increase longevity.

Examples of the present invention are set forth below.

### EXAMPLE 1

### Expansion of Embryonic Stem Cells While Applying Microcurrent

Basal Media was first prepared by aseptically combining 50 mL of MCGS, 440 mL of MSCBM, 10 mL of 200 mM L-Glutamine and 0.5 mL of Penicillin/Streptomycin (25 units penicillin; 25 μg streptomycin). The Basal Media was then supplemented with 10 mL hrLIF/hrFGF, 21 mL nGF/mGF (Atrium Biotechnologies, Inc., Quebec, Canada), 21 mL non-essential amino acids, 30 mL L-Glutamine, 1 mL of 0.1 mM mercaptoethanol, 1.2 mL of 1 mM sodium pyruvate, 90 mL of 20% fetal bovine serum and 400 mg Trizma base. The supplemented Basal Media was adjusted to pH 7.5 with NaOH, filtered through a Nalgene® disposable 0.2 μm filter and aseptically dispensed into Corning 75 mm #2 flasks (20 mL supplemented basal medium per flask).

Frozen human embryonic stem cells were thawed according to standard procedures, and approximately 10⁵ to 10⁶ cells were added to each flask. The flasks were placed on their backs in an incubator with 5% CO₂, and negative and positive electrodes of a microcurrent generator (M.E.N.S.® Microcurrent, Monad Corp., Pomona Calif.) were attached to opposite sides of the flask.

The flasks were then incubated for five days at 37° C. A microcurrent (6 μAmps for 1 to 10 Hz) was applied to the flasks for one hour each day. After five days, the cells were aseptically scraped from the inner surface of the flasks to place the cells into suspension in the medium. The entire contents of each flask was then aseptically aspirated into sterile 50-mL centrifuge tubes and centrifuged at 4000 rpm for 3 to 10 minutes. The supernatant was aseptically aspirated and discarded, leaving the expanded embryonic stem cells at the bottom of the tube.

The cells were gently placed into suspension by aseptically adding 20 mL of 10% DMSO Phosphate buffer to each tube, followed by gentle mixing. The cells were then aliquoted into 1.5 mL tubes and frozen using a Controlled Rate Freezing System, Model 9000 (Gordinier Electronics, Inc., Roseville Mich.). The frozen cells were stored at −15° C.

Four of the cell aliquots were used for quality assurance testing. Microscopic examination of these cells revealed a 99% viability. PCR tests of the cells were negative for Mycoplasma incognitus and others. The cells were also tested for the presence of CD117 using an ELISA assay (Appledale, Essex, U.K.). All four aliquots were positive for CD117, indicating the presence of live hematopoietic stem cells, as shown below in Table 1.

TABLE 1
CD117 ELISA Results for Expanded Embryonic Stem Cells

| Sample | Absorbance at 405 nm |
|---|---|
| Positive Control (Anti mouse- | 3.229 |
| IgM alkaline phosphatase | |
| conjugated—1:1 dilution) | |
| Negative Control (Anti-CD 117— | 0.065 |
| 1:1 dilution) | |
| Aliquot 1 | 3.315 |
| Aliquot 2 | 3.454 |
| Aliquot 3 | 3.826 |
| Aliquot 4 | 3.840 |

### EXAMPLE 2

### Migration of Embryonic Stem Cells to Stressed and/or Cancer Cells

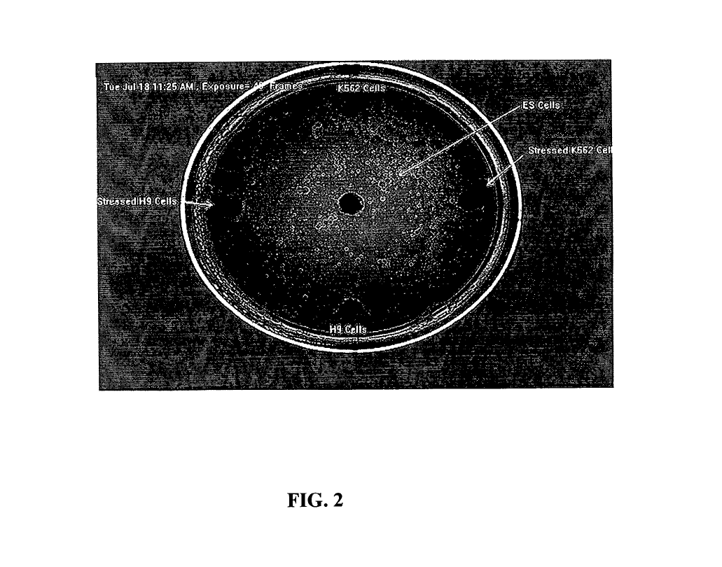

Embryonic stem cells labeled with ethidium bromide were found to migrate preferentially to human cells that had been stressed, causing damage to the cell membrane, or to human cancer cells, or, most preferentially, to stressed human cancer cells. This is shown in FIG. 2.

A petri dish containing a semi-hard 2% agar supplemented with media was prepared by adding 0.67 grams of agarose to 11 mL of 1×TBE buffer, boiling until the agarose was completely melted, adding 20 mL of DMEM and 2 mL of fetal bovine calf serum, and pouring the resulting mix into the petri dish to a depth of 0.5 inches. After the supplemented agarose solidified to a semi-hardened gel, five circular holes were punched into the agar of each plate to form five round wells. As shown in FIG. 2, one hole was punched in the center of the plate and the remaining four were punched around the circumference of the plate, spaced approximately equidistant from each other and equidistant from the center well.

Embryonic stem cells prepared according to the practice of the invention were labeled with ethidium bromide, a fluorescent dye that emits visible light when excited by ultraviolet light, by incubating 1 mL of the cells (approximately 6×10⁶ cells) with 20 μL ethidium bromide for 15 minutes at 37° C. A 100 μL sample of the labeled cells was added to the center well of the prepared petri dish.

Human H9 cells from a CD4+ lymphoblast cell line were grown to a concentration of 5×10⁶ cells/mL. An aliquot of the H9 cells was rapidly frozen and thawed three times to damage the cell membranes (“stressed H9 cells”). A 100 AL sample of H9 (unstressed) cells was added to the bottom well, while a 100 μL sample of stressed H9 cells was added to the left well.

Human K562 cells, an erythroleukemia cell line, were grown to a concentration of 2×10⁶ cells/mL, and an aliquot of these cells was similarly subjected to three cycles of rapid freezing/thawing to damage cell membranes (“stressed K562 cells”). A 100 μL sample of K562 (unstressed) cells was added to the top well, while a 100 μL sample of stressed K562 cells was added to the right well.

The petri dish was placed in an incubator at 37° C. for three days. Following incubation, the plate was exposed to ultraviolet light and photographed. The results are shown in FIG. 2.

The white glow seen in FIG. 2 reveals the presence of the ethidium bromide-labeled embryonic stem cells. As can be seen, the embryonic stem cells migrated outward from the center well during the 3-day incubation period, with a greater number of cells, as reflected by the intensity of the fluorescent label, concentrating at or near stressed H9 and the K562 cells, and, particularly, near the stressed K562 cells. Relatively fewer labeled embryonic stem cells accumulated at or near the unstressed H9 cells, which, as normal cells, serve as a negative control.

In the following examples, the use of embryonic stem cells to treat human patients with a wide range of diseases, disorders or other conditions is described. In all cases, patients were treated in countries outside of the United States.

### EXAMPLE 3

### Treatment of a Patient with AIDS using CCR5/CCR5 Embryonic Stem Cells

A 33-year old female living in Europe (Patient X) with late stage AIDS was given a prognosis of only a few days to live. Patient X presented with a sequela of HIV-related infections, including non-Hodgkins lymphoma, Candida albicans, Kaposis sarcoma (in the lung, mouth, skin and vagina), and Trichomonas, as well as hepatic insufficiency, as manifested by elevated liver enzymes, extreme dehydration notwithstanding intravenous administration of saline solution, chronic diarrhea, and extreme weakness. Patient X had a CD4 count of 35, and CD8 count of 586, and a CD4/CD8 ratio of 0.06. (A normal CD4/CD8 ratio is higher than 1.)

Embryonic stem cells (hematopoietic and neuronal) were isolated from an aborted fetus determined to be homozygous CCR5-def. Approximately 40 million hematopoietic stem cells were diluted with distilled water to 6 cubic centimeters and were given to Patient X via intravenous injection, along with subcutaneous injections of neuronal stem cells (approximately 40 million cells diluted with distilled water to 6 cubic centimeters). Both embryonic human and sheep thymus was also administered. Within six weeks of this treatment, Patient X was free of all HIV-associated infections, her liver enzyme levels had returned to normal, she no longer experienced diarrhea, her strength was close to normal and she was eating normally with a 9 pound weight gain. Her 6-week post-treatment CD4 count was 410, and her CD8 count was 512, resulting in a CD4/CD8 ratio of 0.8.

### EXAMPLE 4

### Treatment of Sickle Cell Disease with Expanded Embryonic Stem Cells

Hematopoietic stem cells (approximately 10 million to 40 million cells), pre-screened to ensure that they contained normal beta-globin genes (i.e., did not carry the mutation known to result in sickle cell disease) were injected intravenously into Patient Y, who had been previously diagnosed with sickle cell disease and who was displaying symptoms of the disease at the time of injection. Approximately 10 million to 40 million pre-screened neuronal stem cells were also administered by subcutaneous injection.

Prior to the injections, Patient Y's hemoglobin electrophoresis results indicated a 98.6% HGB S variant, consistent with sickle cell disease. Following injection of the hematopoietic cells, Patient Y became asymptomatic for sickle cell disease, and hemoglobin fractionation tests conducted five months later indicated a drop in the percentage of the HGB S variant to 64.2%, with a HGB A concentration of 35.8%, a pattern typically found in patients with sickle cell trait (i.e., clinically normal heterozygous carriers of the sickle cell mutation). This indicates that Patient Y is now producing both normal (HGB A) and sickle cell (HGB S) hemoglobin variants, consistent with the disappearance of sickle cell disease symptoms.

In each of the following Examples 5 through 23, patients were treated with both an intravenous injection of about 10 million to about 100 million hematopoietic stem cells and a subcutaneous injection of about 10 million to about 80 million neuronal stem cells. For both cell types, the cells were first diluted with distilled water to a final volume of 6 cubic centimeters per dose.

### EXAMPLE 5

### Treatment of Downs Syndrome

Hematopoietic and neuronal stem cells were administered to a Downs Syndrome patient. Following the injections, the patient experienced significant improvement of cognitive function.

### EXAMPLE 6

### Treatment of Leukemia

In combination with traditional chemotherapy, hematopoietic and neuronal stem cells were injected intravenously and subcutaneously, respectively, into a patient suffering from leukemia. The patient experienced a complete remission of the disease.

### EXAMPLE 7

### Treatment of Systemic Lupus Erythematosus Rheumatoid Arthritis

Following the injection of hematopoietic and neuronal stem cells, patients with systemic lupus erythematosus rheumatoid arthritis exhibited a significant reduction of symptoms. In particular, these patients experienced a significant reduction in pain and rash.

### EXAMPLE 8

### Treatment of Osteoporosis

Patients with osteoporosis were treated with injections of hematopoietic and neuronal stem cells. Following the injections, the rate of bone formation in these patients exceeded the rate of bone resorption, resulting in increased bone mass and density.

### EXAMPLE 9

### Use of Embryonic Stem Cells to Treat Immune Failure Following Chemotherapy Overdose

Patient Z was diagnosed with ovarian cancer and treated with conventional chemotherapy. An accidental overdose of the cytostatic drug, in an amount six times the lethal dose (900 mg cisplatine and 145 mg toxol), effectively destroyed Patient Z's immune system, leaving her with a white count of 0.

Embryonic hematopoietic stem cells were administered to Patient Z following the overdose via intravenous injection. Within 12 days, Patient Z's white count had returned to normal. As of two years following treatment, Patient Z remains asymptomatic of cancer with normal blood counts.

### EXAMPLE 10

### Treatment of Multiple Sclerosis

A patient with multiple sclerosis was injected intravenously with hematopoietic stem cells and subcutaneously with neuronal stem cells. Prior to the injections, the patient was confined to a wheelchair and exhibited plaques and diminished hand strength. Following the injections, the patient's hand strength improved, the plaques diminished, and the patient regained the use of his legs.

### EXAMPLE 11

### Treatment of Amyotrophic Lateral Sclerosis

Patients with amyotrophic lateral sclerosis were treated with both hematopoietic and neuronal stem cells. Following the injections, the progression of the disease slowed, stopped, or in come cases, reversed, enabling some patients previously confined to a wheelchair to walk again.

### EXAMPLE 12

### Treatment of Cerebral Palsy

Following injections of hematopoietic and neuronal stem cells, patients with cerebral palsy displayed significant improvement in cognitive and motor skills.

### EXAMPLE 13

### Use of Embryonic Stem Cells to Treat Brain Damage

Patients with brain injuries experienced significant improvements in cognitive and motor skills following injection of hematopoietic and neuronal stem cells. Patients also demonstrated positive changes in their single photon emission computed tomography (SPEC) scans (including actual brain growth) and their electroencephalographic (EEG) recordings.

### EXAMPLE 14

### Treatment of Parkinson's Disease

Following injections of hematopoietic and neuronal stem cells, patients with Parkinson's disease displayed significant improvement in cognitive and motor skills, and experienced relief of tremor, stiffness and Parkinsonian mask.

### EXAMPLE 15

### Use of Embryonic Stem Cells to Treat Early Stage Alzheimer's Disease

Administration of hematopoietic and neuronal stem cells to patients exhibiting the symptoms of early stage Alzheimer's disease resulted in a complete disappearance of all symptoms.

### EXAMPLE 16

### Use of Embryonic Stem Cells to Treat Stroke Patients

Stroke patients treated with hematopoietic and neuronal stem cells exhibited improvement in both cognitive and motor function.

### EXAMPLE 17

### Use of Embryonic Stem Cells to Treat Other Hypoxic Disorders

Embryonic stem cells have also been used to treat patients with other hypoxic disorders, such as victims of near drownings or birth trauma. In these cases, administration of hematopoietic and neuronal stem cells resulted in significant improvement in cognitive and motor skills.

### EXAMPLE 18

### Treatment of Autism

Following injections of hematopoietic and neuronal stem cells, autistic patients showed a significant increase in cognitive abilities and interpersonal interactions.

### EXAMPLE 19

### Treatment of Type I Diabetes

Diabetic (Type I) patients treated with hematopoietic and neuronal stem cells experienced a 50% decrease in insulin demand and showed improvement in secondary complications of diabetes such as circulatory deficits.

### EXAMPLE 20

### Use of Embryonic Stem Cells Following Surgical Procedures

Patients undergoing a variety of surgical procedures were treated post-surgery with hematopoietic and neuronal stem cells via intravenous and subcutaneous injection, respectively. These patients exhibited an accelerated healing rate, decreased internal fibrosis and reduced scarring.

### EXAMPLE 21

### Treatment of Cancer with Embryonic Stem Cells

Several patients with various types of cancer have been treated with embryonic stem cells. Following injection of both hematopoietic and neuronal stem cells, cancer patients experienced positive results in both morbidity and mortality rates.

### EXAMPLE 22

### Treatment of Depression

Patients diagnosed with depression were treated with hematopoietic and neuronal stem cells. Following the injections, the patients experienced significant changes in affect, and, in some cases, the depression completely cleared.

### EXAMPLE 23

### Cosmetic Uses of Embryonic Stem Cells

Embryonic stem cells are also used for cosmetic purposes. Injection of both hematopoietic and neuronal stem cells produced significant changes in skin tone and diminished wrinkles. For patients that were not significantly overweight, administration of embryonic stem cells resulted in a decrease in fat and an increase in lean muscle. Several patients treated with embryonic stem cells have reported improvement in athletic performance.

### EXAMPLE 24

### Treatment of Retinal Vein Thrombosis

A patient with retinal vein thrombosis was treated with hematopoietic stem cells via retro bulbar injection. Prior to treatment, the patient was blind in that eye. Following the injection of the hematopoietic stem cells (approximately 10×10⁶ cells), vision in that eye was restored to 20/50, although the patient's peripheral vision remained suboptimal.

### EXAMPLE 25

### Use of Embryonic Stem Cells to Treat a Non-Healing Bone Fracture

A patient with a fibular fracture that had failed to heal for six years was treated with an injection of pluripotent hematopoietic embryonic stem cells (approximately 10×10⁶ cells) directly into the subperiostium of the bone. Within two months, the fracture had healed, presumably due to differentiation of the injected embryonic stem cells to osteoblasts producing bone growth at the site of the fracture.

### EXAMPLE 26

### Mucosal Lesions

Hematopoietic pluripotent stem cells were administered by intravenous injection to a patient with ulcers. Within two weeks, the ulcers were completely cleared.

### EXAMPLE 27

### Use of Embryonic Stem Cells to Stimulate Creativity

Another effect of the administration of embryonic stem cells reported by several patients receiving the treatment is the stimulation of creativity and clarity of thought. Many patients, following treatment with both hematopoietic stem cells (approximately 10 million to 100 million cells, injected intravenously) and neuronal stem cells (approximately 10 million to 80 million cells, injected subcutaneously) report an increased ability to concentrate and focus their thoughts, as well as enhanced memory.

In addition to the above described examples of the actual use of embryonic stem cells to treat a variety of diseases and disorders, it is anticipated that embryonic stem cells will also provide therapeutic benefit in the treatment of patients with a wide variety of diseases.

For example, it is anticipated that patients with hemophilia can be given injections of embryonic stem cells known to contain a functional gene for the Factor VIII blood protein, either by intravenous injection or injection directly into the spleen or liver. Optionally, the injection may be preceded by aplasia of the patient's bone marrow. Once injected, the embryonic stem cells differentiate into mature, Factor VIII producing cells.

Similarly, it is anticipated that patients with heart disease can be treated with injections of embryonic stem cells into cardiac muscle, where the cells differentiate into cardiac muscle cells, replacing damaged heart tissue. Other types of organ damage can likely be treated in the same manner.

Other diseases believed to be treatable with embryonic stem cells include Mucosviscidosis (fybrocystic disease of the pancreas), viral and bacterial infections (by providing embryonic stem cells to supplement the patient's immune system), hematological diseases, and a range of mental disorders, including schizophrenia. It is further anticipated that regular administration of embryonic stem cells will increase longevity, by providing a constant source of new cells to replace damaged cells or cells with chromosomal damage.

The above descriptions of exemplary embodiments are illustrative of the present invention. Because of the variations, which will be apparent to those skilled in the art, however, the present invention is not intended to be limited to the particular embodiments described above. The scope of the invention is defined in the following claims.

What is claimed is:

1. A method for treating patients with a condition, disease or disorder selected from the group consisting of sickle cell disease, Downs syndrome, leukemia, systemic lupus erythematosus rheumatoid arthritis, osteoporosis, multiple sclerosis, amyotrophic lateral sclerosis, cerebral palsy, brain damage, Parkinson's disease, early stage Alzheimer's disease, stroke, hypoxic disorders, autism, type I diabetes, cancer, depression, hemophilia, heart disease, muscoviscidosis, and mycoacidosis, comprising the steps of:
administering hematopoietic stem cells to the patient via intravenous injection; and
administering neuronal stem cells to the patient via subcutaneous injection.

2. The method of claim 1, wherein about 10 million to about 100 million hematopoietic stem cells and about 10 million and to about 80 million neuronal stem cells are administered to the patient.

3. The method of claim 1, wherein the hematopoietic stem cells and the neuronal stem cells are prepared by introducing embryonic stem cells into a chemically defined culture medium containing a growth factor, applying a microcurrent to the culture medium containing the stem cells, and incubating the culture medium containing the stem cells at a temperature from about 34° C. to about 42° C.

4. The method of claim 1, wherein the hematopoietic stem cells and the neuronal stem cells are extracted from a human embryo.

5. The method of claim 1, further comprising the step of performing aplasia of the patient's bone marrow prior to administering the hematopoietic stem cells and the neuronal stem cells.
